(19) United States
(12) Patent Application Publication
(10) Pub. No.: US 2008/0091469 A1
(43) Pub. Date: Apr. 17, 2008

(54) System for prescribing customized medical procedures

(76) Inventors: Lee Allen, Camas, WA (US); Jeff Jackson, Camas, WA (US); Travis Wilhelmsen, Vancouver, WA (US); Nathan Pryor, Vancouver, WA (US); Lou Graham, Chicago, IL (US); David A. Little, San Antonio, TX (US); Kerri M. Sebring, York, PA (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/582,620

(22) Filed: Oct. 17, 2006

(51) Int. Cl. G06F 19/00; A61B 5/00

(52) U.S. Cl. 705/3

(57) ABSTRACT

A system for prescribing a customized medical procedure that is to be performed on an individual patient.

### CROSS-REFERENCE TO RELATED APPLICATIONS

Not applicable.

### BACKGROUND OF THE INVENTION

The present disclosure relates to an interactive system for prescribing a customized medical procedure that is to be performed on an individual patient.

Many medical procedures must be customized either to physical conditions that are particular to an individual patient, or to the aesthetic or financial wishes of an individual patient. For example, a dental patient may need a bridge after having a tooth knocked out. This particular restoration procedure requires that the missing tooth be replaced with what is called a pontic—a dental replacement that has no real tooth beneath it—and that the two adjoining teeth be crowned so as to provide the proper support to the pontic necessary when chewing, for example. Rather than obtaining and inserting the pontic and the two adjoining crowns as separate components, dentists will typically insert a bridge into the patient's mouth, which is a dental implant that includes the pontic and the two crowns already fabricated as a single piece. When ordering the bridge to insert into the patient's mouth, the dentist not only must find one that has the appropriate dimensions that fit the patient's mouth, but also must find one that has a shading that matches the teeth already in the patient's mouth. Both of these instances involve customizing the implant to the physical condition of the patient. In addition, there are many alternative materials from which dental implants are made, e.g. gold, silver, ceramic, etc. While the choice of material is not a matter of necessity, the patient may desire one or the other for aesthetic or financial reasons, or both.

Ordinarily, when making these determinations, the dentist or other analogous medical provider will use an ad-hoc, practitioner-specific procedure to determine and notate the required or desired specifications unique to the particular patient. The variable nature by which this vital information is obtained often becomes problematic, not only for the laboratory or supplier who must provide a suitable product o the patient, but also for the patient who might ultimately receive a product or medical procedure not as satisfactory as desired. For example, and continuing the example cited in the preceding paragraph, while one dentist may photograph a patient's mouth and write measurements on the photograph so that a bridge may be specially fabricated that physically fits the patient's mouth, another dentist might make an elastomeric impression of the patient's mouth so as to provide a laboratory with far more detailed information from which custom implants can be fabricated. Similarly, the dentist may use any one of a number of varying shade guides to notate the natural coloring of a patient's teeth, and to which an implant should be matched. From this information, it is often difficult for a laboratory to fashion an implant that is optimally matched to the physical dimensions and coloring of a patient's mouth. In fact, it is not uncommon for an implant prescription to request nothing more than the restoration type desired, e.g. bridge, crown, etc., and a shade designation. Absent providing a laboratory with complete information about a patient's mouth, a practitioner risks receiving an implant that does not fit the patient (resulting in either the need to delay the procedure for a better match to be fabricated), discomfort if the procedure is performed, or even a second corrective procedure.

Furthermore, unless the patient is already particularly well-versed in the procedure to be received, the patient depends heavily on the practitioner to provide whatever information is necessary for the patient to adequately balance cost and aesthetic considerations when selecting materials, or making other choices pertinent to a given medical procedure. If insufficient information is conveyed, the practitioner risks an aesthetic outcome with which the patient is unhappy.

Even the most meticulous medical practitioner cannot completely eliminate these risks. Some practitioners, no matter how technically capable in the procedures they perform, are nonetheless deficient in other relevant skills. For example, as noted by many trade publications, many dentists lack the visual acuity to match the color of a patient's teeth to a preset shade on a shade guide. This task is complicated by the fact that teeth are not typically of a uniform shade, so that a dentist will often have to specify different shades, not only for different implants in the bridge, for example, but also for different portions of each individual implant, as natural shading often varies even within a particular tooth. Furthermore, many medical practitioners may not be well versed in the relative strengths and weaknesses of medical products, particularly if those products are new and/or their relative strengths and weaknesses occur over a longer term than the practitioner treats a given patient.

What is desired, therefore, is an improved system for medical practitioners to customize medical procedures and services to suit particular patients.

### BRIEF DESCRIPTION OF THE SEVERAL VIEWS OF THE DRAWINGS

FIG. 1 shows a block diagram of an exemplary interactive prescription generation system for a forthcoming medical procedure.

FIGS. 2-8 each show respective interactive displays of the exemplary system of FIG. 1.

### DETAILED DESCRIPTION OF PREFERRED EMBODIMENT

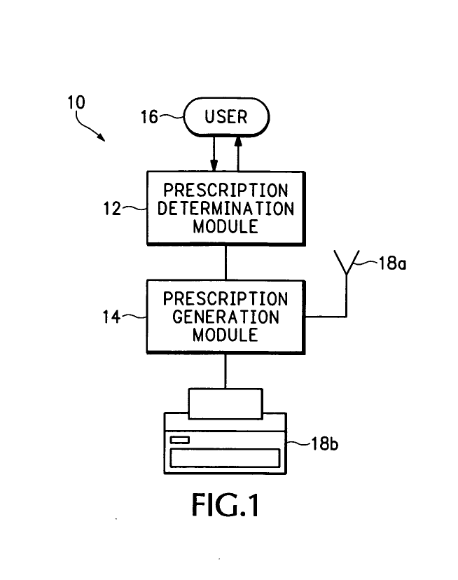

FIG. 1 shows an exemplary interactive system 10 that assists a medical practitioner in communicating relevant information regarding a pending medical procedure for a patient, not only to any appropriate third-party medical service or medical equipment provider, but also to a patient who needs such information to make informed decisions regarding the information communicated to the third party medical service. Specifically, the disclosed system 10 preferably includes an interactive, iterative prescription determination module 12, a prescription generation module 14, and one or more communication devices, such as the wireless transmitter 18a or a printer 18b to communicate, to a third-party medical service, any prescriptions determined by the module 12 and generated by the module 14.

The term “prescription” when used in this specification refers broadly to any instructions by a medical practitioner specific to a patient and given to a third-party medical provider that furnishes either services or products in accordance with those instructions. A prescription, for example, could refer to instructions for making or furnishing dental implants, prosthetic limbs, breast or face implants, pharmaceuticals, contact lenses, or any other analogous product. Furthermore, though FIG. 1 draws the prescription determination module 12 and the prescription generation module 14 as separate elements, that separation is merely conceptual; an actual embodiment of the depicted system may incorporate features of both into the same interactive display, subroutine, etc. Finally, the depicted communication devices 18a and 18b are illustrative only, in that many other similar devices, such as a modem, telephone, etc. may provide the same functionality.

The prescription determination module 12 preferably provides a user, which could include a medical practitioner, the practitioner's patient, or both, with one or more relevant options pertaining to the prescription. For example, if the prescription is for a dental implant, the prescription determination module 10 preferably includes options to select or input a type of implant, a type and location of tooth the implant is to be applied to, the medical procedure the implant is to be used in, the size of the implant if more than one implant is to be used in the procedure, the relative spacing between implants, the shading of each implant, etc. Furthermore, the prescription determination module 12 is preferably both iterative and interactive, meaning that the module 12 permits a practitioner and patient, not only to repeatedly select different choices for each of the available options, but to receive feedback as to the consequences of those choices so that, through an iterative procedure, both the practitioner and the patient may arrive at a prescription that represents the optimal combination of available variables in the prescription.

Once a prescription has been determined that satisfies both the practitioner and the patient, the prescription generation module 14 automatically generates a prescription that conforms to the determined prescription, which includes the choices made by the practitioner and/or patient. The prescription may preferably also be automatically sent to the relevant third-party medical provider, either by wireless communication of the prescription, by modem, or other communication channel. Alternatively, the prescription may be printed and delivered to the third-party provider by mail or facsimile, or may be displayed on a monitor and communicated to the third-party provider by telephone.

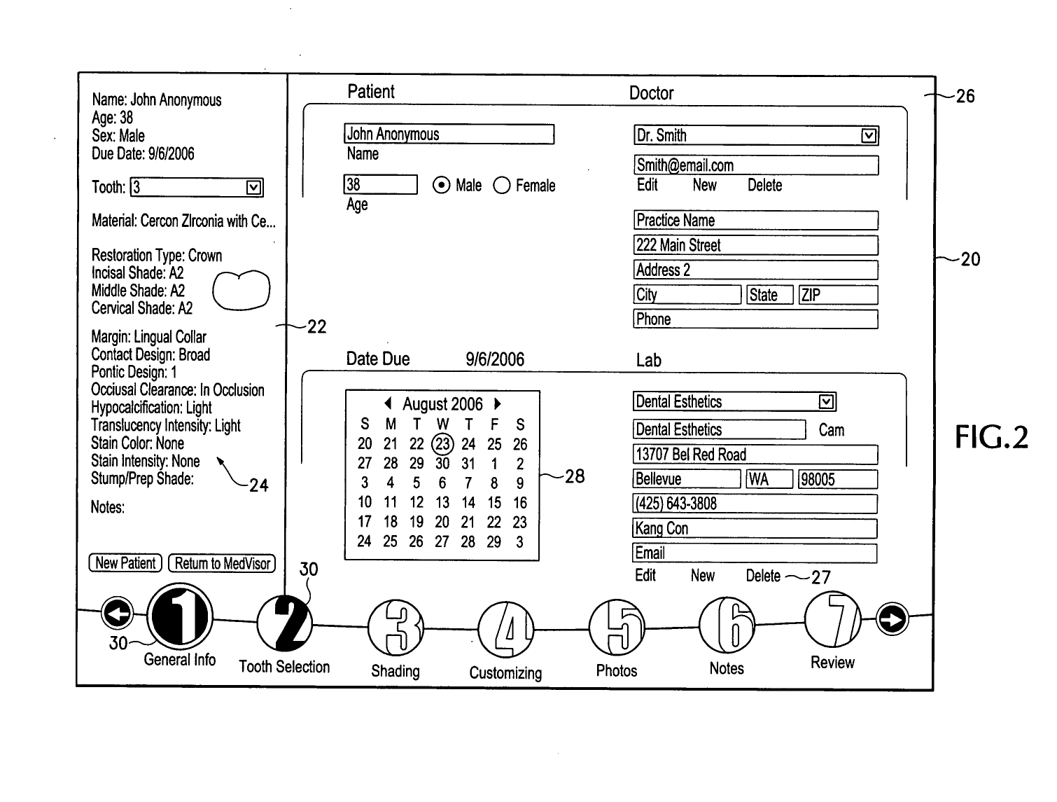

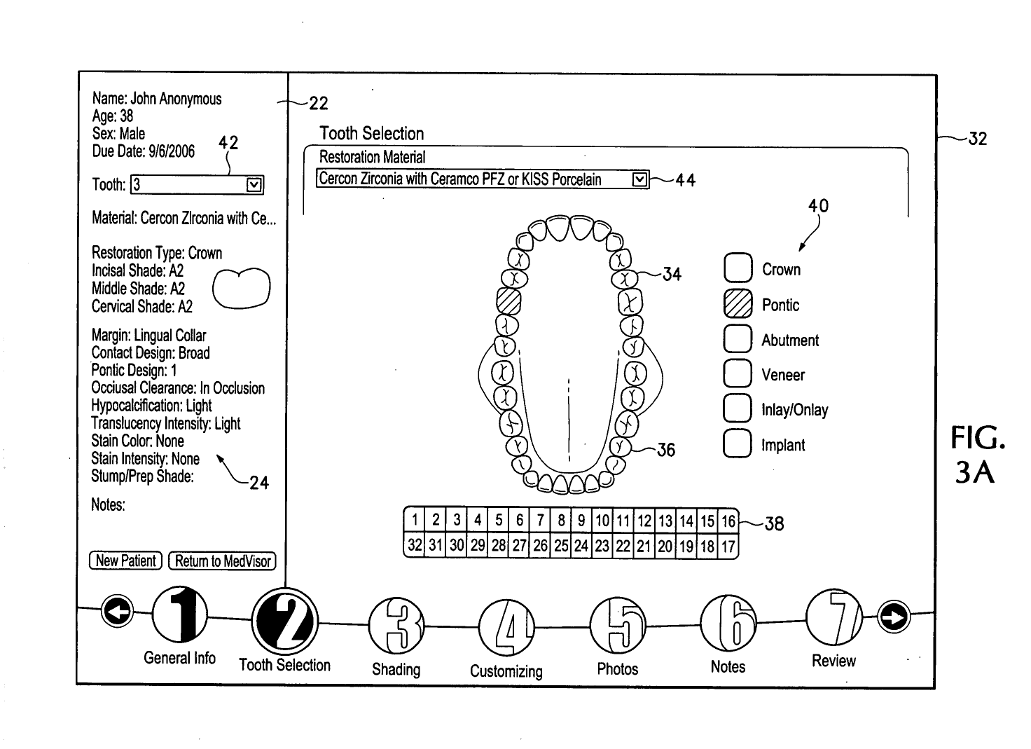

FIGS. 2-8 show an exemplary interactive system 10 specific to determining and generating a prescription for dental implants. The exemplary system 10 may preferably be embodied in an executable computer program resident on a tablet PC or other electronic device having an interactive display, such as a laptop computer, personal computer, PDA, stand-alone kiosk similar to those for automatically ordering books, music, etc. Though the system 10 specifically shown in FIGS. 2-8 was written in the Director/Lingo programming language, it may instead be alternatively written in any number of other programming languages, such as JAVA, C++, visual basic, or any other appropriate programming code that is now in existence or that may later be developed. Furthermore, the present system 10 may be embodied in a physical storage medium, such as a compact disk, internet server, etc. from which the system 10 may be loaded/downloaded onto a device for executing the instructions in the program. The disclosed system 10 could be resident on a server accessible by a network connection from another electronic device that has a display.

The disclosed system 10 may be a stand-alone product, or may alternatively be incorporated into a more comprehensive package. For example, a co-inventor of the present application has filed patent application Ser. No. 11/082,233, disclosing an interactive patient education system and which is incorporated herein by reference, to which the disclosed system 10 may be combined, or added as a module or component. Also, though FIGS. 2-8 show an illustrative system 10 intended to be used by a dental practitioner when prescribing dental implants, it should be understood that the system 10 may be more comprehensively embodied to prescribe not only other types of prescription products, but may be used for multiple prescription types. For example, a cosmetic surgeon may not only perform dental reconstructions but also provide a number of other medical services such that the practitioner might routinely prescribe a variety of products such as dental implants, cheek implants, prosthetic limbs, etc. Such a practitioner may then use a single system 10 capable of determining and generating prescriptions for each of the products the practitioner might order.

Referring to FIG. 2, a first interactive display 20 of the exemplary system 10 may include a panel 22 that displays cumulative summary information 24 related to the prescription being determined and later generated. The summary information 24 is preferably completed through practitioner/patient interaction through the panels such as 26. For example, panel 26 is an interactive display by which general statistical or demographic information related to the prescription may be entered, such as the patient's name, age, gender, etc., as well as the name and contact information of both the prescribing practitioner and the third-party provider filling the prescription. The panel 26 may also present a user with a field to enter the date the prescription is due. Where appropriate, these fields may include writable text fields for names, mutually exclusive check boxes for information such as gender, and drop down menus containing pre-entered information likely to be repeated among a plurality of patients, e.g. practitioner's or third-party provider's names, etc. For convenience, selection of an option from a drop down menu may automatically fill in contact or other information pertinent to that selection, such as the address and phone number of a selected laboratory. An interactive button menu 27 may allow for manual additions, deletions, or edits to this pre-entered information. Furthermore, the system 10 may allow for appropriate information, such as available labs, to be updated automatically via a network connection when a new provider enters a market or an existing provider offers additional products. The display 20 preferably includes a calendar 28 by which a due date for the prescription is selected. This calendar is exemplary only, as other embodiments of the system 10 may include other menus by which the date may be entered, such as a drop-down menu, text field, etc. The display 20 also preferably includes navigation icons 30 by which a user may navigate among the various exemplary displays depicted in FIGS. 2-8.

As information is entered into the panel 26, the summary prescription information 24 in the panel 22 is preferably updated to reflect the information entered. The panel 22 is preferably visible in each of the subsequent displays accessed through the navigation icons 30.

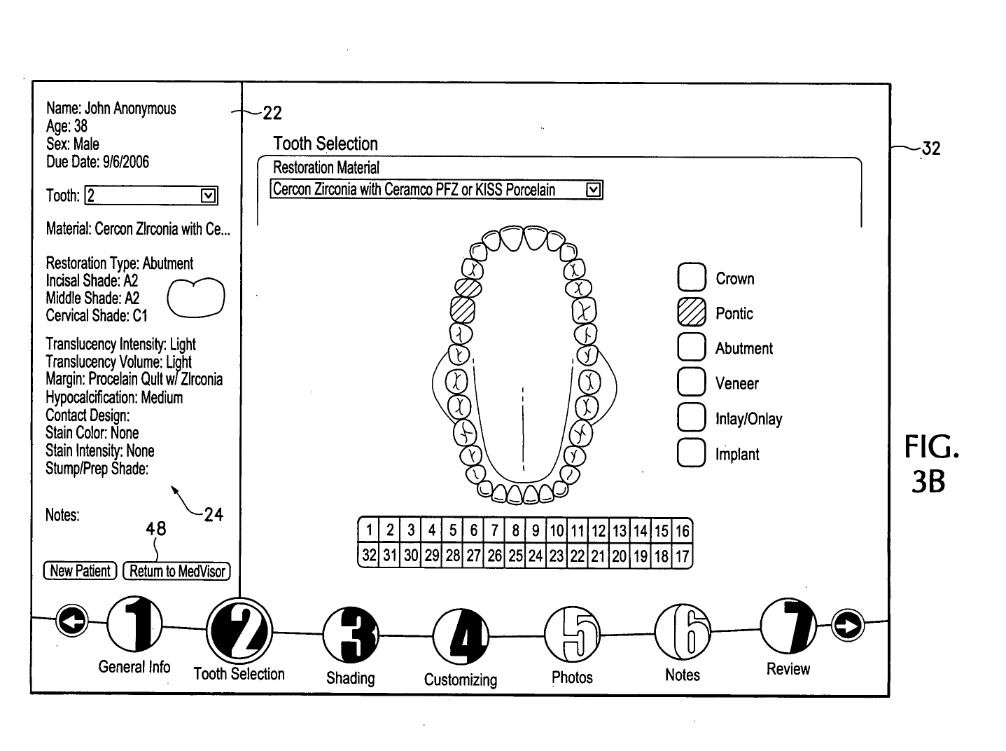

Referring, for example, to FIGS. 3A and 3B, a tooth selection display 32 also includes the panel 22, which displays general statistical and demographic information entered in the display 20. From the display 32, a user may select features of a prescription unique to the medical procedure to be applied to the patient using an interactive selection process. For example, the display 32 may preferably include a pictorial representation 34 of a mouth with a full complement of teeth 36. Each of the teeth 36 may be separately represented in a numbered table 38 and medical prescription product options may be displayed in a panel 40. For example, the interactive display 32 may provide a drag-and drop menu in the panel 40 by which a user may drag any one of the available prescription product options, i.e. crown, pontic, abutment, veneer, inlay/onlay, and implant, onto a particular tooth that is to receive the product being prescribed. Thus, in FIG. 3, a user has dragged the pontic product over tooth number 3 and in FIG. 3A, a user has dragged a pontic over each of teeth numbers 3 and 4 and an abutment over teeth 1 and 5. Also, the display 32 may also include a drop-down menu 44 by which a material for the products may be selected, such as Cercon Zirconia ceramic material, or gold, etc. Each of these selections is preferably reflected in the summary panel 22, such that a tooth to which a product has been applied becomes available for selection in a drop-down menu and selection of that tooth displays relevant prescription information, such as the restoration product type, material, etc. Furthermore, the panel 22 is preferably intelligent, in the sense that selection of certain combinations of product/teeth combinations will be recognized by the system 10 as being certain named procedures. For example, if a user uses the drag and drop interactive display to select a pontic over a tooth surrounded on each side by an abutment, the system 10 will recognize the procedure as a bridge and denote it as the restoration type in the panel 22 so that the practitioner need not enter it manually.

Another feature of the system 10 is that the drop-down menu selections for the material types of the selected products may be cross referenced to the drop-down menu selections for the available labs, so that certain materials may be automatically eliminated from or grayed out in the drop down menu 44 if a selected lab does not provide that type of material, and vice versa. This may be an invaluable service to a practitioner. For example, a practitioner, in the initial display 20, may elect to leave the lab field blank until the patient chooses a material type, at which point the system automatically winnows out labs that do not provide that material. In this manner, the practitioner need not keep track which labs offer what materials, because the system 10 does it for the practitioner. This functionality may easily be incorporated into the system 10 by the inclusion of a simple database that relates materials and labs, which is referenced by the system when compiling and refreshing the drop-down menus or other interactive selection mechanisms.

Referring to FIGS. 3B and 3C, the system 10, may provide yet another invaluable patient education feature in regarding the consequences of certain choices, such as the choice of material for a particular restoration. The system 10 may include animations 46 that display typical time-lapse sequences of how particular restorations and other procedures will look or develop over time. For example, when a person ages, gums will typically recede to expose portions of teeth and restorations not earlier visible. Thus, if a patient using the system selects the option of a gold restoration, for example, the practitioner can then select, by pressing a button 48 for example, a display of an animation sequence showing how that restoration might typically look a number of years into the future. The animation sequences (or still images) may be played, paused, forwarded and rewound, or otherwise navigated using a control toolbar 50. The patient may then repeat the process with one or more different restoration materials, to see comparative animations of those alternative materials and thereby make a more informed decision as to an appropriate material to prescribe. Alternatively (or additionally), the system 10 could include still photographs that show the same sequence, or show still photographs of actual restorations that use the selected materials. The animation sequences could be accompanied by audio or text that explains what is shown.

This capability of the system 10 facilitates a novel business method by manufacturers or other providers of third-party medical products and services who wish to communicate the strengths of their products or services relative to their competitors. For example, since gums recede with age, exposing more implant material, a manufacturer of ceramic restoration material such as Cercon Zirconia may wish to comparatively advertise its product as being better than gold, silver, etc. in that it will look more natural, even over an extended period of time. Using conventional advertising methods, such a manufacturer would either expend a great deal of money buying television or print advertisements targeting the end-user consuming public—a very inefficient method given that it is directed sweepingly at the viewing or reading audiences—or alternatively, by dispatching a sales representative to practitioners so as to educate them about the asserted relative strengths of the product, and thereby relying entirely upon the practitioner to either inform their patients about the relative strength of the product, or simply prescribe the product based on their own opinions. Obviously, each of these conventional methods is relatively inefficient in communicating the desired message to the person or persons who will be using the product.

The system 10, however, permits a third-party provider of medical equipment to develop one or more effective presentations that show the strength of its products relative to those of one or more competitors with a greater assurance that, not only will the presentation be viewed by a targeted audience, but that the presentation will be viewed precisely when it matters the most—during a consultation with a medical practitioner for a medical procedure that will use the very product promoted. In one embodiment, a manufacturer of a medical prescription could be the provider of the system 10 to a practitioner for the practitioner's use in prescribing a medical product. As a specific example, a manufacturer of ceramic dental implants could provide a system 10 that comprehensively permits a customer-practitioner to prescribe a wide variety of dental restorations using materials supplied by, not only that manufacturer, but competing manufacturers, as well. However, the animation sequences, still photographs, etc. that are available within the system 10 may show the manufacturer's product in a favorable light with respect to those of competitors by highlighting either the relative strengths of the manufacturer-provider's products, the relative weaknesses of competing products, or both.

In a second embodiment, a provider of the system 10 could charge a fee to one or more manufacturers of medical prescription material, who would then be permitted to supply the animation sequences, still photographs etc. that show their products in a positive light. This latter embodiment would be particularly suitable for a system 10 that determines and generates prescriptions for a wide variety of medical prescription products.

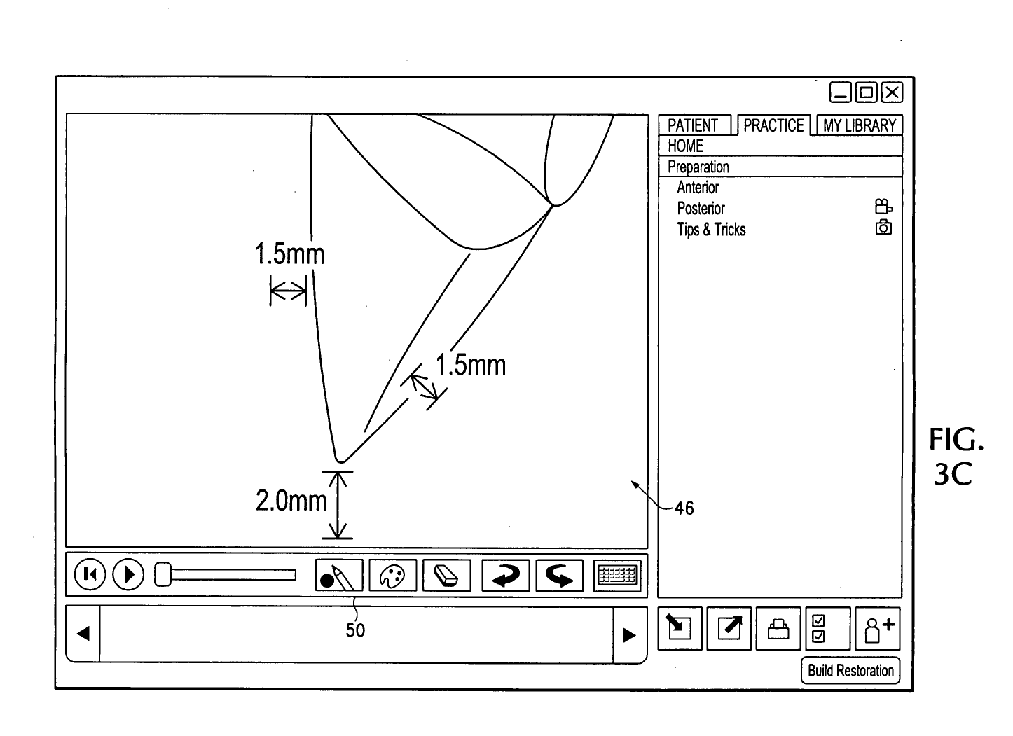

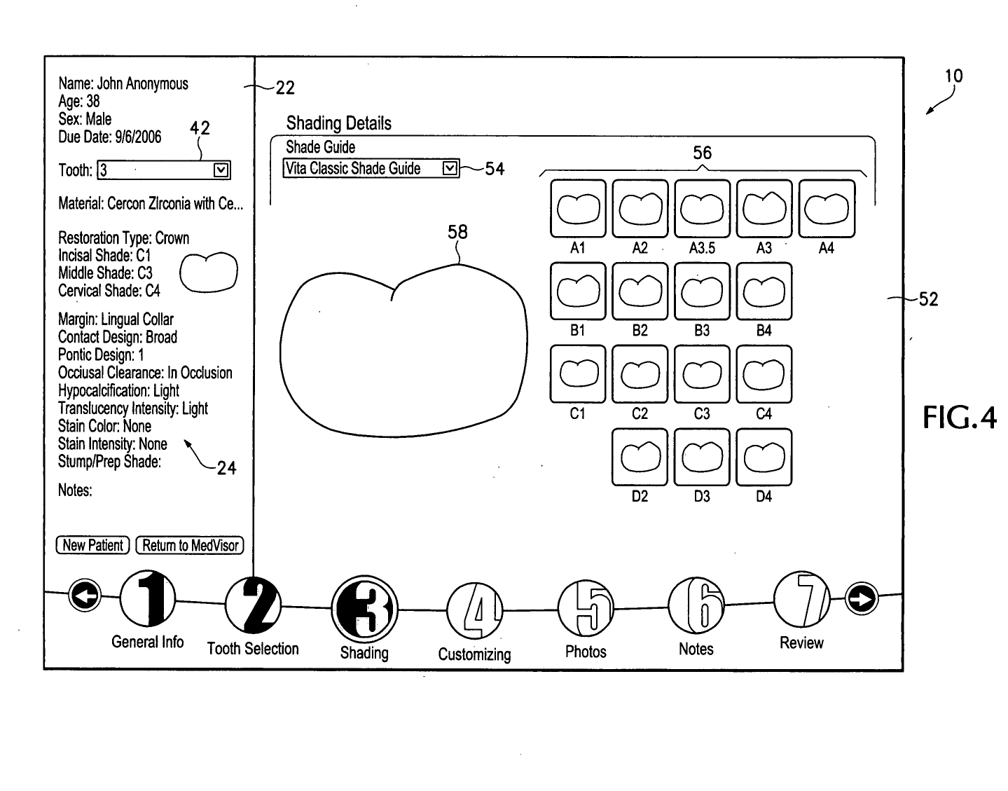

Referring to FIG. 4, once the practitioner has indicated the teeth to which a restoration will apply and the system 10 has thereby been provided with the type or types of implants to be prescribed, the practitioner may use a shading interface 52 to apply an appropriate shading to each tooth in a prescribed implant. As stated earlier, the system 10 has preferably recognized, without user input, the type of implant required so that, by navigating with drop down menu 42 in the panel 22, a practitioner may individually select one of the teeth in the prescribed implant for shading, and the system 10 will identify the restoration type to be applied, e.g. “crown” as shown in FIG. 4. Furthermore, the practitioner may choose an appropriate shade guide from a drop-down menu 54 or other substitute interface. Like the laboratory drop-down menu shown in FIG. 2, the shade guides included in the drop-down menu may be updated, either by an Internet download, or by update software provided by disc, etc.

Selection of an available shade guide from the drop-down menu 54 will preferably cause the tooth shadings 56 particular to that shade guide, along with their corresponding identifiers, e.g. A1, B4, etc., to be displayed in the panel 52. An enlarged representation 58 of the tooth selected from the drop-down menu 42 of the panel 22 may also be displayed. The tooth shadings 56 are preferably drag-and-drop, i.e., the practitioner may drag a selected one of the shadings 56 over the representation 58, which will then display the shading dropped upon it. If the practitioner is dissatisfied with that shading, another may be dropped onto the representation 58, and that particular shading will replace the earlier one. Furthermore, the enlarged representation 58 may be segregated into multiple areas, each of which is capable of being separately shaded. For example, the practitioner may use the drag-and-drop interface to separately select the incisal shade (upper region of the tooth 58), the middle shade, and the cervical shade (lower region of the tooth 58) so as to more accurately duplicate the natural coloring of a person's tooth. As these selections are made and updated, the selections are displayed in the panel 22 for verification by the practitioner. Once a particular tooth of the implant has been shaded to the practitioner's and patient's satisfaction, the drop-down menu 42 may be used to select the tooth in the implant, if any remains, for shading.

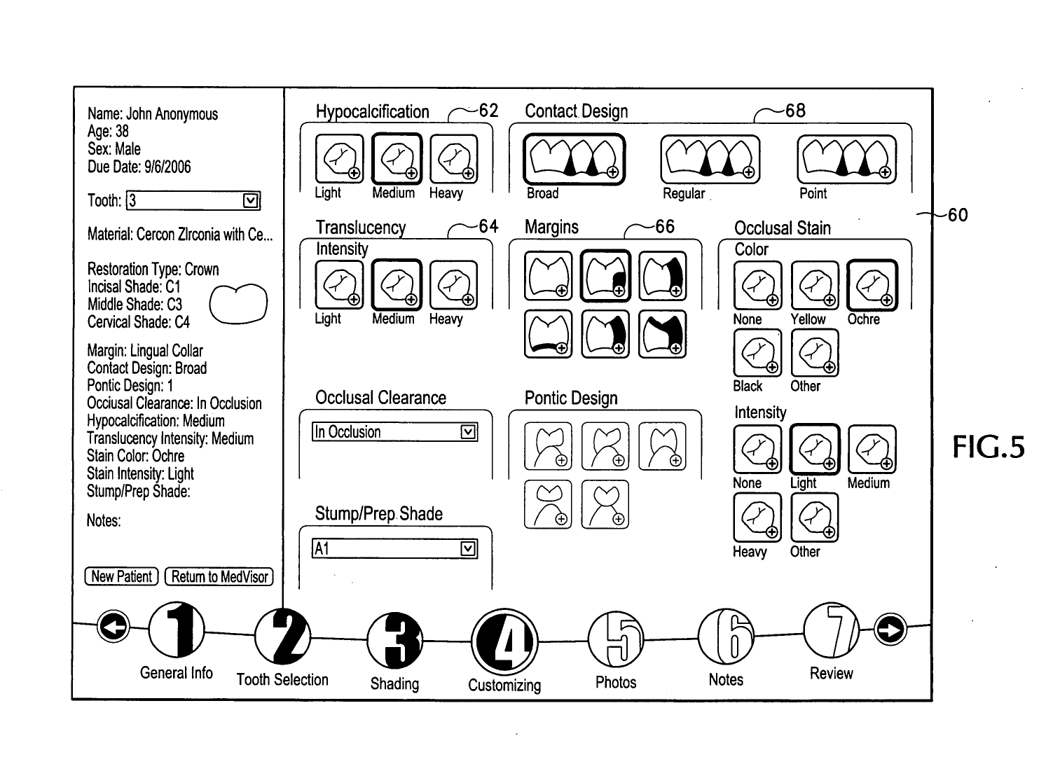

Referring to FIG. 5, certain prescription materials may have additional available options, for which additional display 60 may be used to permit a practitioner to select among them. For example, dental implant prescriptions often specify additional details about the desired product, such as hypocalcification 62, translucency 64, margins 66 and contact design 68. Unlike the shading options shown in display 52, these additional options shown in display 60 may not be applicable to all dental implants for example, or stated more broadly, for all prescriptions of a type for which the system 10 is capable of determining and generating. Accordingly, the system 10 is preferably capable of disabling any options in the display 60 that are not available for a certain implant, for a certain material, etc. This can bee seen in FIG. 5, which has shaded out, i.e. disabled, the options of contact design 68 and margins 66, which are not applicable to the restoration type of a crown. Also, as can be seen in FIG. 5, selection of an available option is again reflected in the panel 22, and any particular interface may be used to select the available options, e.g. drop-down menu, button selection of pictorial representations, etc.

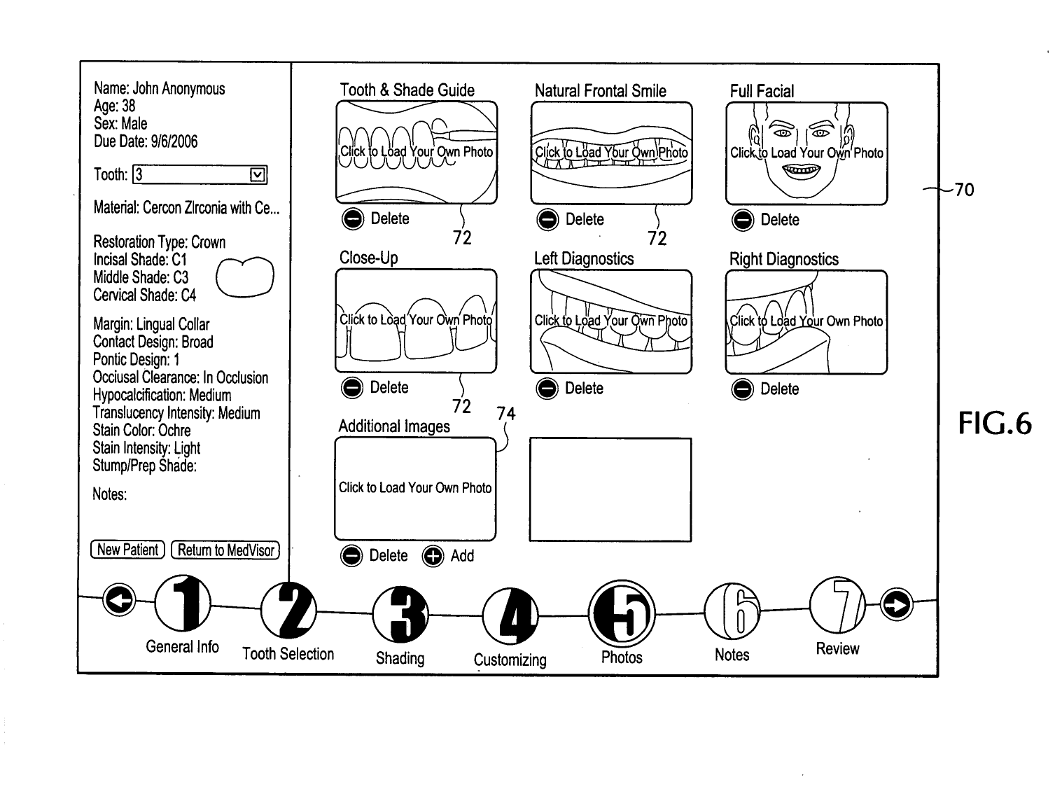

Referring to FIG. 6, where appropriate, the system 10 may include a display 70 that permits a practitioner to attach actual photographs of the patient to the prescription, when such photographs would be helpful to the third-party that fills the prescription. The display 70 may include pre-labeled, individually selectable regions 72 within which digital photos may be appended to the prescription, For example, a digital photograph of a patient's mouth may be appended to a prescription by clicking or otherwise selecting one of the preset regions, which then brings up a standard navigation menu within which the appropriate file may be found and selected. Preferably, the system 10 includes a hard drive or other permanent storage upon which the photographs of the patient may be loaded. For example, to facilitate the ability to browse to, and insert, such photographs, the system 10 may be loaded on a computer having a hard drive that stores the photos. More preferably, the system 10 may operate on a device having a USB, compact flash, CD or DVD ROM drive, or other port that is capable of reading digital photos from a mobile storage device, and writing them to the hard drive so as to append to the prescription. Alternatively, if the system lacks a hard drive or other permanent storage, the system preferably has sufficient memory to retain a number of such digital photographs so as to electronically transfer or print the photographs when the prescription is finished, or alternatively electronically transfer or print the photographs along with the prescription while the mobile storage is still connected to the particular electronic device from which the prescription is determined and generated. As can be further seen in FIG. 6, the display 70 may preferably include a region 74 not pre-labeled so that the prescribing practitioner may attach one or more miscellaneous images that either do not fall into any of the pre-labeled categories, or are additional images of one of the pre-labeled categories. To this end, the region 74 may include a button or other mechanism that permits a user to indicate whether an image will be added are deleted.

Though the display 70 shows interactive icons or regions 72 from which digital two-dimensional photographs may be appended to the prescription, the system 10 may either substitute, or additionally include, regions by which three-dimensional visuals of the patient, or any other analogous three-dimensional image, may be appended. For example, a dental practitioner may make a three-dimensional impression of the patient's mouth. This impression may then be imaged in three dimensions and packaged into a digital file, from which an editing program can construct any desired two-dimensional view, and/or from which a viewing utility can show the image in three dimensions by, e.g. a rotatable interface. This described alternate embodiment of the system 10 would include the ability to append such multi-dimensional imagery of that impression to the prescription, in the same manner as the two dimensional photographs are appended.

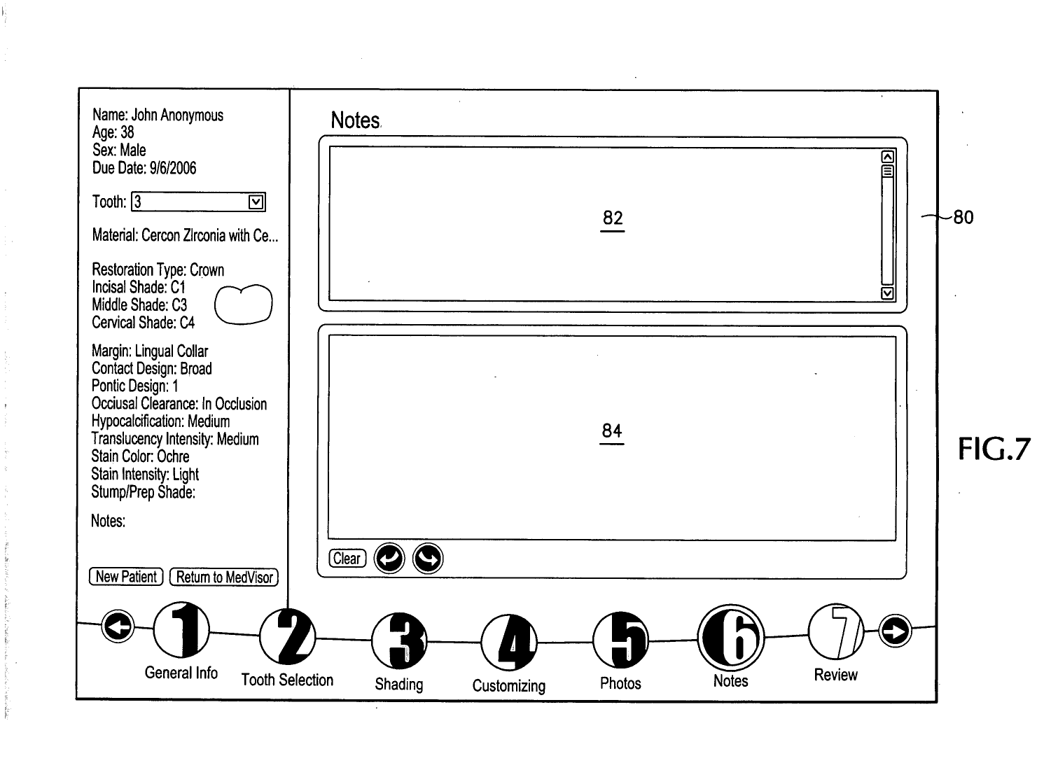

Referring to FIG. 7, the system 10 may include a display 80 into which a practitioner may enter any notes to be appended to the prescription. Entry of these notes may be accomplished by a keyboard or a computer-readable pen, or any other desired means. In a preferred embodiment, the display 80 may include a first portion 82 to add text that may be entered with either a computer keyboard, or a graphic keyboard that recognizes taps from a magnetic pen. Preferably, any text entered in this first portion 82 is reflected in the panel 22 at the left portion of the display 80. Furthermore, the display 80 may include a second portion 84 into which a practitioner may enter handwritten drawings and/or notations to append to the prescription.

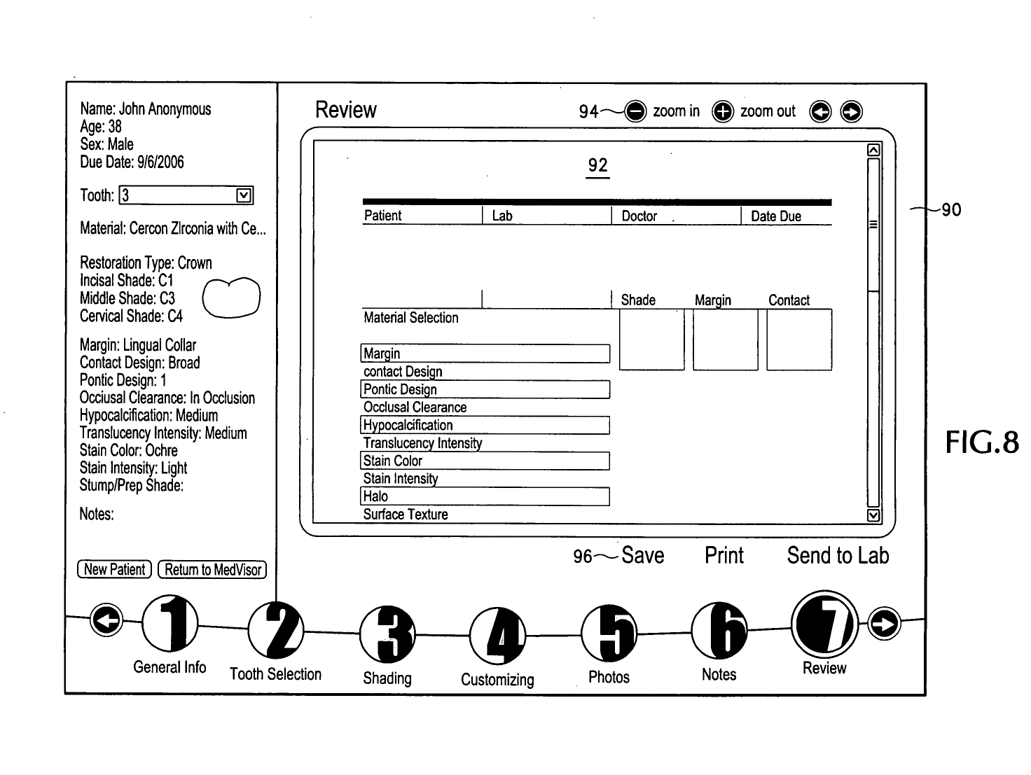

Referring to FIG. 8, the system 10 may include a display 90 having a panel 92 from which the practitioner may review the prescription prior to it being generated and sent to the third-party that fills the prescription. Preferably, though the prescription information in the panel 92 includes information summarized in the panel 22 at the left portion of the display 90, the information in the panel 92 is more comprehensive. Furthermore, the panel 90 preferably includes navigation buttons such as 94 that assist in reviewing the information in the panel 92. The display 90 also preferably includes options 96 to save the prescription to disc, print the prescription, or send the prescription to the party identified in the display 20 as the party filling the prescription.

The terms and expressions that have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims that follow.

I/We claim:

1. A system comprising:
(a) a prescription determination module capable of receiving input for a prescription parameter having a plurality of available options and having an interface permitting a user to iteratively select different said ones of said available options where selection of at least one of said available options results in an indication of the effect of said option on a patient receiving said prescription; and
(b) a prescription generation module capable of selectively generating a prescription indicating a currently selected said available option for said prescription parameter.

2. The system of claim 1 where said indication is at least one of a visual and an auditory indication.

3. The system of claim 1 having a plurality of said prescription parameters.

4. The system of claim 3 where a first one of said prescription parameters is a dental implant type and said plurality of available options associated with said dental implant type include at least one of a crown, a pontic, an abutment, a veneer, an inlay/onlay, and an implant.

5. The system of claim 4 where a second one of said prescription parameters is implant shading and said plurality of available options associated with said dental implant type are selected from standardized dental shading guides.

6. The system of claim 3 where selection of one or more particular options associated with a first prescription parameter eliminates at least one options associated with a second parameter, and said system disables eliminated said options.

7. The system of claim 1 including a database that is automatically updatable without manual input.

8. An automated method for generating a prescription having a parameter with a plurality of associated available options, said method comprising:
(a) receiving a first manual input selecting a first said available option;
(b) indicating, without manual input, the effect of said first available option on a patient receiving said prescription;
(c) receiving a further manual input selecting another said available option;
(d) indicating, without manual input, the effect of said another available option on a patient receiving said prescription;
(e) selectively repeating steps (c) and (d); and
(f) in response to a user input, automatically generating a prescription that specifies a currently selected available option with its associated prescription parameter.

9. The method of claim 7 where said steps of indicating the effects of selected said available options on a patient receiving said prescription is at least one of a visual and an auditory indication.

10. The method of claim 7 including the step of repeating steps (a) through (e) for a plurality of said prescription parameters.

11. The method of claim 10 where a first one of said prescription parameters is a dental implant type and said plurality of available options associated with said dental implant type include at least one of a crown, a pontic, an abutment, a veneer, an inlay/onlay, and an implant.

12. The system of claim 11 where a second one of said prescription parameters is implant shading and said plurality of available options associated with said dental implant type are selected from standardized dental shading guides.

13. The system of claim 10 where selection of one or more particular options associated with a first prescription parameter eliminates at least one options associated with a second parameter, and including the step of disabling eliminated said options.

14. The system of claim 8 including the step of automatically updating, without manual input, a database from which said available options are presented to a user and associated with said prescription parameter.

15. A business method comprising the step of advertising a prescription product by providing a medical practitioner who makes prescriptions that include said prescription product with an electronic device, said electronic device containing an advertisement to be presented to a patient who is to receive a said prescription from said practitioner, said advertisement to be presented to said patient during an interactive portion of a consultation between said practitioner and said patient.

16. The business method of claim 15 where said electronic device is used by said practitioner in the furtherance of the practitioner's business.

17. The business method of claim 16 where said electronic device is used by said practitioner to generate said prescription.

18. The business method of claim 15 including the step receiving solicitations from marketers of said prescription products to provide a said advertisement for inclusion within said electronic device.
